(19) United States
(12) Patent Application Publication
(10) Pub. No.: US 2014/0200615 A1
(43) Pub. Date: Jul. 17, 2014

(54) Anti-Displacement Coil Spring-Type Spine Stabilization Device

(71) Applicant: Paonan Biotech Co., Ltd., Taipei City (TW)

(72) Inventor: Chung-Chun Yeh, Taipei City (TW)

(21) Appl. No.: 13/936,538

(22) Filed: Jul. 8, 2013

(30) Foreign Application Priority Data
Jan. 11, 2013 (TW) 102200771

(51) Int. Cl. A61B 17/70

(52) U.S. Cl. CPC A61B 17/7028
USPC 606/250; 606/255

(57) ABSTRACT

An anti-displacement coil spring-type spine stabilization device includes: a coil spring member made of a spiral spring wire having multiple continuous loops, the coil spring member having the form of an elongated bar; two connection members securely connected with two ends of the coil spring member, opposite end faces of the two connection members being respectively formed with two axially extending sockets; and at least one elastic bar disposed in the coil spring member and axially extending through the coil spring member with two ends of the elastic bar respectively extending into the sockets of the connection members. The coil spring member is made of the spiral spring wire so that two ends of the coil spring member are naturally formed with threads, which can be directly screwed on the connection members without further mechanically processing. This simplifies the manufacturing process to lower the manufacturing cost.

### RELATED APPLICATION

This application claims the benefit of a Taiwanese patent application, 102200771, filed on Jan. 11, 2013, the specification of which is incorporated here by this reference.

### BACKGROUND OF THE INVENTION

1. Field of the Invention

The present invention relates generally to an anti-displacement coil spring-type spine stabilization device, and more particularly to a spine stabilization device including a coil spring member made of a spiral spring wire having multiple continuous loops. The coil spring member has the form of an elongated bar. Two connection members are locked on or elastically latched with two ends of the coil spring member. Opposite end faces of the two connection members are respectively formed with two sockets. At least one elastic bar is disposed in the coil spring member and axially extends through the coil spring member with two ends of the elastic bar respectively extending into the sockets of the connection members.

2. Description of the Related Art

It is known that when the intervertebral disc between two vertebras of the spine is damaged to compress the nerve, a patient will feel sore and painful. In the case of serious injury, currently, the patient is cured by means of a fusion surgery to relieve the patient from the uncomfortableness. In the fusion surgery, a metal rod member is locked on the lesion vertebra and two vertebras on upper and lower sides of the lesion vertebra. That is, multiple vertebras are fixedly fused to protect the lesion vertebra from being reinjured by compression force and relieve the patient from the pain and uncomfortableness.

However, the metal rod member used in the fusion surgery cannot be elastically bent and tensioned. Therefore, the fixedly fused vertebras are prevented from swinging or bending again. Under such circumstance, the two normal intervertebral discs on upper and lower sides of the fusion surgery range must bear all the action force applied to the spine when the patient moves.

In order to improve the above problem, U.S. Pat. No. 8,080,038 discloses a double-layer spring rod, which is fixed between multiple vertebras to release the lesion vertebra and relieve the patient from the pain. Moreover, the double-layer spring rod is tensile and bendable so that the vertebras of the spine can swing or bend along with the movement of the patient in the moving direction. However, when the patient jumps or runs, an abrupt upward and downward action force will be applied to the double-layer spring rod to axially compress the same. As a result, the lesion vertebra is likely to be compressed again to make the patient feel painful. In addition, as shown in FIG. 1, the double-layer spring rod 60 is made of spiral spring wires having multiple loops. The adjacent loops can hardly bear greater radial action forces F, F′ so that the loops are very likely to deflect and displace under the radial action forces F, F′ as shown in FIG. 1. That is, the spine stabilization device of U.S. Pat. No. 8,080,038 can hardly protect the vertebras from radial action force. In this case, when the patient moves or bears an external force, the vertebras P1, P2, P3 locked with the double-layer spring rod 50 are apt to relatively deflect and displace. As a result, the lesion vertebra may be further injured.

To solve the above problem, U.S. Publication No. US2007/0049937 A1 [application Ser. No. 11/509,544] discloses a rod-shaped implant element 1. As shown in FIGS. 6 to 13 of this patent, the middle section of the rod-shaped implant element 1 is formed with helical opening 10, whereby the rod-shaped implant element 1 is flexible. In addition, closure members 13, 14 are locked or riveted at two ends of the elongated rod-shaped implant element 1. A core 12 is fitted in the elongated rod-shaped implant element 1. In this embodiment, the core 12 is able to avoid deflection. However, the elongated rod-shaped implant element 1 has a very thin wall. Accordingly, in manufacturing, in the case that the elongated hollow rod-shaped implant element 1 is directly cut with helical opening 10, the processing force often causes deformation of the hollow rod-shaped implant element 1. To avoid the deformation, generally a solid bar is first cut with the spiral groove and then one end of the bar body is milled and bored. The bore passes through the spiral groove to form a perforated helical opening 10 on the rod-shaped implant element 1. Then, two ends of the rod-shaped implant element 1 are respectively processed to form threaded hole for locking with the closure members 13, 14. Therefore, the rod-shaped implant element 1 needs to be mechanically processed four times. This complicates the manufacturing process and leads to increase of cost. Moreover, the rod-shaped implant element 1 is implanted in human body so that the rod-shaped implant element 1 must be free from any burred or sharp edge. In this case, it is more difficult to process the rod-shaped implant element 1.

U.S. Pat. No. 7,329,258 discloses another spine stabilization device in which the first sprint member 2 is made of a solid bar by means of mechanical processing and cutting as in U.S. Patent No. US2007/0049937 A1. Similarly, the first sprint member 2 needs to be mechanically processed many times. This also complicates the manufacturing process and leads to increase of cost and needs to be improved.

In U.S. Publication No. US2007/0049937 A1 and U.S. Pat. No. 7,329,258, the rod-shaped implant element 1 or the first sprint member 2 are formed with spiral groove by means of a cutter. The spiral groove has a certain width so that the rod-shaped implant element 1 or the first sprint member 2 is axially compressible. This is unsuitable for a patient with injured spine due to compression. Neither of the above two patents is able to effectively resist against axial action force. That is, neither of the above two patents can effectively support the injured spine. This needs to be improved.

### SUMMARY OF THE INVENTION

It is therefore a primary object of the present invention to provide an anti-displacement coil spring-type spine stabilization device, which is securely fixable on one side of a spine by multiple pedicle screws. The spine stabilization device includes: a coil spring member made of a spiral spring wire having multiple continuous loops, the coil spring member having the form of an elongated bar; two connection members, each connection member having a connection section, the connection sections being securely connected with two ends of the coil spring member, opposite end faces of the two connection members being respectively formed with two axially extending sockets; and at least one elastic bar disposed in the coil spring member and axially extending through the coil spring member with two ends of the elastic bar respectively extending into the sockets of the connection members. Even if the coil spring member is axially tensioned to a maximum allowable length or bent to a maximum curvature, the two ends of the elastic bar are still positioned in the sockets of the connection members. The coil spring member is made of a spiral spring wire. Therefore, two ends of the coil spring member are naturally formed with threads, which can be directly screwed on the corresponding spiral grooves of the connection members. Alternatively, the threads can be elastically engaged with the annular grooves of the connection members. According to the above arrangement, after the coil spring member is formed, the coil spring member can be securely connected with the connection members without being further mechanically processed. This facilitates the manufacturing process to lower the manufacturing cost.

In the above anti-displacement coil spring-type spine stabilization device, it is impossible to pull and multistage deflect the elastic bar axially extending through the coil spring member. Therefore, the coil spring member is prevented from being greatly displaced under radial action force.

In the above anti-displacement coil spring-type spine stabilization device, the pedicle screw has a base seat and a screw shank protruding from a bottom of the base seat for screwing into the spine. The base seat is formed with a chuck. At least one rib is formed on the chuck for engaging between two adjacent loops of the coil spring member. A plug is locked in the chuck to tightly hold the coil spring member between the chuck and the plug. The pedicle screw is not included in the scope of the present invention. Any other conventional pedicle screw is also applicable to the present invention without limitation.

In the above anti-displacement coil spring-type spine stabilization device, the pitch between the adjacent loops of the coil spring member is equal, unequal, or nearly zero. That is, the compressible distance of the coil spring member is near zero. In this case, the lesion vertebras can be effectively supported and released. Moreover, the injured vertebras or intervertebral discs of the spine are protected from being reinjured due to over-compression.

In the above anti-displacement coil spring-type spine stabilization device, the connection member connected with the end of the coil spring member has a connection section. A rod body axially extends from one end of the connection section. The rod body is locked and connected with a pedicle screw. An end face of the connection section is axially recessed to form at least one socket. The rod body of the connection member is formed with a shaft hole for locking on a fixing shaft. Accordingly, the angle contained between the connection member and the coil spring member is adjustable in accordance with the requirement of installation of the anti-displacement coil spring-type spine stabilization device on the cervical vertebras and the occipital.

In the above anti-displacement coil spring-type spine stabilization device, the connection member has a rod body. A connection section axially protrudes from each of two ends of the rod body for connecting with the end of the coil spring member. The end face of the connection section is axially recessed to form at least one socket. One end of the elastic bar is fitted in the socket. The sockets of the connection sections at two ends of the rod body can communicate with each other. In this case, the elastic bar can pass through the connection members and axially pass through multiple coil spring members. In application, the pedicle screw is locked on the rod body between the two connection sections of the connection member so as to securely fix the coil spring member on one side of the spine.

In the above anti-displacement coil spring-type spine stabilization device, the connection section of the connection member is formed with spiral groove in adaptation to the spiral angle of the coil spring member. Accordingly, the connection member can be snugly locked on the end of the coil spring member. Alternatively, the connection section of the connection member is formed with an annular groove. The loops of the coil spring member are partially elastically latched in the annular groove.

In the above anti-displacement coil spring-type spine stabilization device, the connection member is made of metal material, alloy material, plastic material or complex material. The material of the connection member is not limited.

In the above anti-displacement coil spring-type spine stabilization device, the elastic bar is made of metal, carbon fiber, plastic or complex material such as PEEK. The material of the elastic bar is not limited. The elastic bar can be straight, curved or angled to meet the curve of human spine.

In the above anti-displacement coil spring-type spine stabilization device, the outer ends of the connection members are formed with drive sections for a wrench or a screwdriver to drive. Accordingly, a wrench or a screwdriver can be used to drive the connection member to make the connection section tightly screwed in the end of the coil spring member.

In the above anti-displacement coil spring-type spine stabilization device, the coil spring member is enclosed in a plastic or rubber sleeve to prevent growing human muscle tissue from infiltrating into the coil spring member of the spine stabilization device.

In the above anti-displacement coil spring-type spine stabilization device, the opposite sides of the adjacent loops of the spiral spring wire are formed with raised section and recessed section engageable with each other. Accordingly, the adjacent loops of the coil spring member are stacked with the raised sections and the recessed sections engaged with each other so as to avoid radial displacement of the adjacent loops.

In the above anti-displacement coil spring-type spine stabilization device, the coil spring member is composed of one single layer of continuous loops or multiple layers of continuous loops.

In the above anti-displacement coil spring-type spine stabilization device, two ends of the elastic bar are movably fitted in the sockets of the two connection members. Alternatively, one end of the elastic bar is fixed in the socket of one connection member, while the other end of the elastic bar is movably fitted in the socket of the other connection member.

In the above anti-displacement coil spring-type spine stabilization device, in application, the pedicle screw can be directly locked on any section of the coil spring member. The coil spring member is composed of multiple continuous loops and can be flexibly bent, tensioned and twisted. Also, the elastic bar axially extending through the coil spring member is elastically bendable along with the movement of the spine. Therefore, when the spine of a patient equipped with the present invention moves, the coil spring member of the present invention can swing or stretch along with the movement of the vertebras of the spine to simulate the movement of a normal spine tissue. This can relieve the patient from uncomfortableness.

In the above anti-displacement coil spring-type spine stabilization device, the coil spring member is formed of a spiral spring wire. After formed, the multiple loops of two ends of the coil spring member will naturally define threaded holes or form outer threads, which can be snugly screwed on the corresponding spiral grooves of the connection members. Alternatively, the loops can be directly elastically engaged with the annular grooves of the connection members. Therefore, the installation of the spine stabilization device is facilitated. Moreover, after the coil spring member is formed, the coil spring member can be securely connected with the connection members without being further mechanically processed many times (such as cutting with the spiral groove, milling or tapping). This facilitates the manufacturing process to greatly lower the manufacturing cost of the present invention. Apparently, the present invention is advantageous over U.S. Publication No. US2007/0049937 A1 and U.S. Pat. No. 7,329,258.

The present invention can be best understood through the following description and accompanying drawings, wherein:

### BRIEF DESCRIPTION OF THE DRAWINGS

FIG. 1 is a side view showing that the conventional spine stabilization device is deflected under relative action force;

FIG. 2 is a perspective exploded view of the present invention;

FIG. 3 is a perspective assembled view of the present invention;

FIG. 4 is a sectional view according to FIG. 3;

FIG. 5 is a perspective view showing the installation of the present invention and the pedicle screws;

FIG. 6 is a side view showing the present invention is installed on one side of the spine;

FIG. 7 is a sectional view showing that the present invention is elastically bent and deformed;

FIG. 8 is a sectional view showing that the coil spring member of the present invention is composed of multiple layers of continuous loops;

FIG. 9 is a side view showing a second embodiment of the connection member of the present invention;

FIG. 10 is a side view showing a third embodiment of the connection member of the present invention;

FIG. 11 is a side view showing a fourth embodiment of the connection member of the present invention;

FIG. 12 is a side view showing a fifth embodiment of the connection member of the present invention;

FIG. 13 is a side view showing a sixth embodiment of the connection member of the present invention;

FIG. 14 is a perspective view showing that multiple connection members are connected with multiple coil spring members of the present invention;

FIG. 15 is a perspective sectional view showing the cross section of the coil spring member in a second aspect;

FIG. 16 is a perspective sectional view showing the cross section of the coil spring member in a third aspect; and

FIG. 17 is a perspective sectional view showing the cross section of the coil spring member in a fourth aspect.

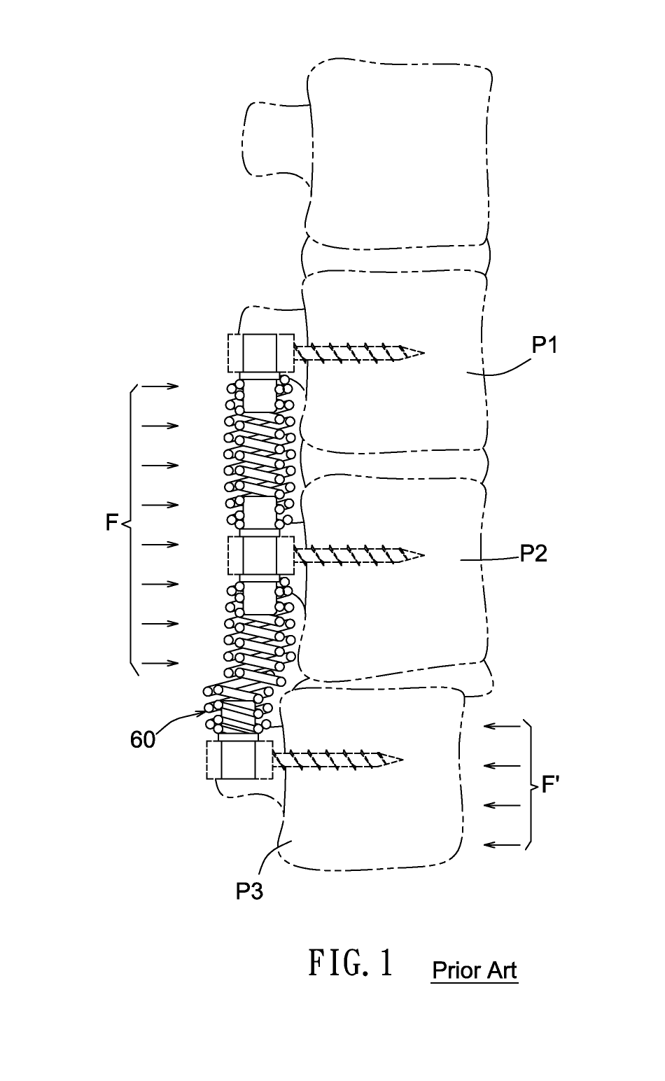

### DETAILED DESCRIPTION OF THE PREFERRED EMBODIMENTS

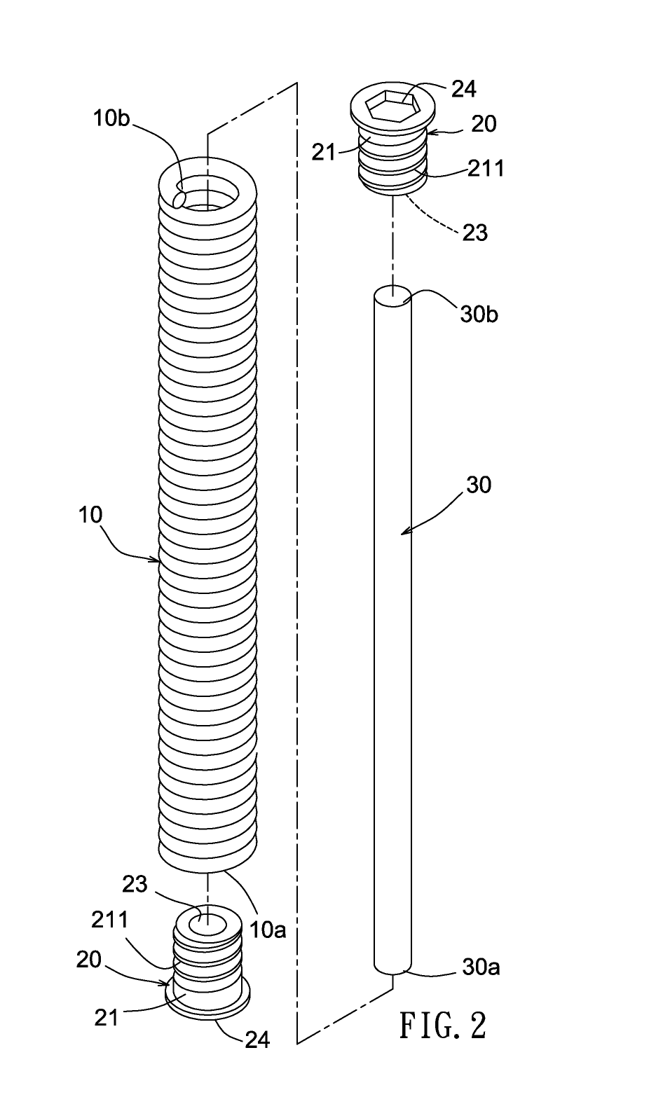

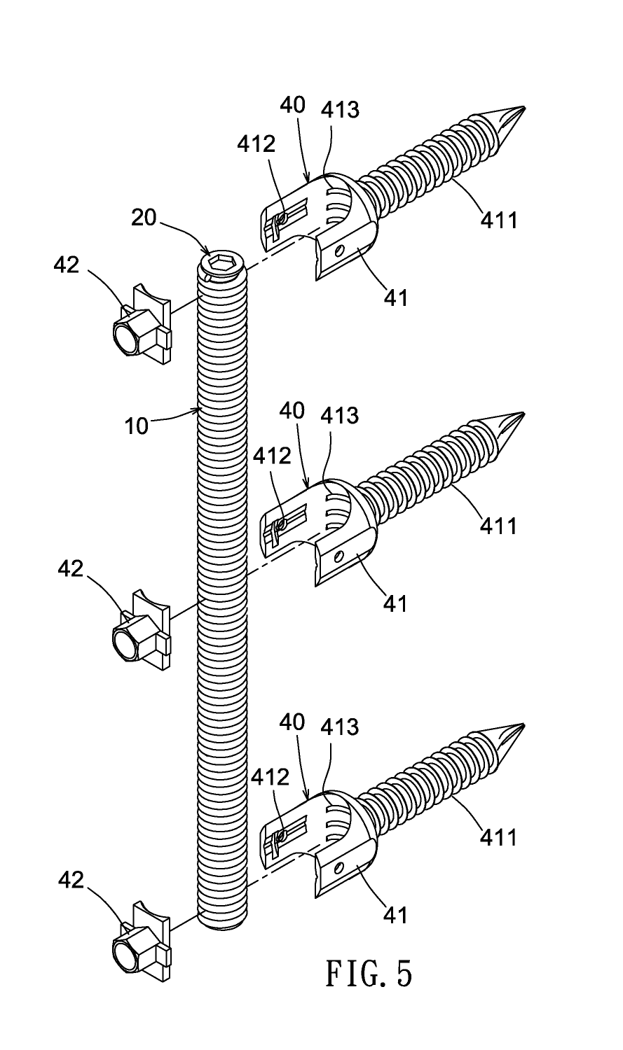

Please refer to FIGS. 2 to 7. The anti-displacement coil spring-type spine stabilization device of the present invention can be securely fixed on one side of a spine 50 by multiple pedicle screws 40. The spine stabilization device of the present invention includes a coil spring member 10. Two connection members 20 are respectively secured to two ends of the coil spring member 10. An elastic bar 30 is disposed in the coil spring member 10 to axially extend through the coil spring member 10 between the two connection members 20.

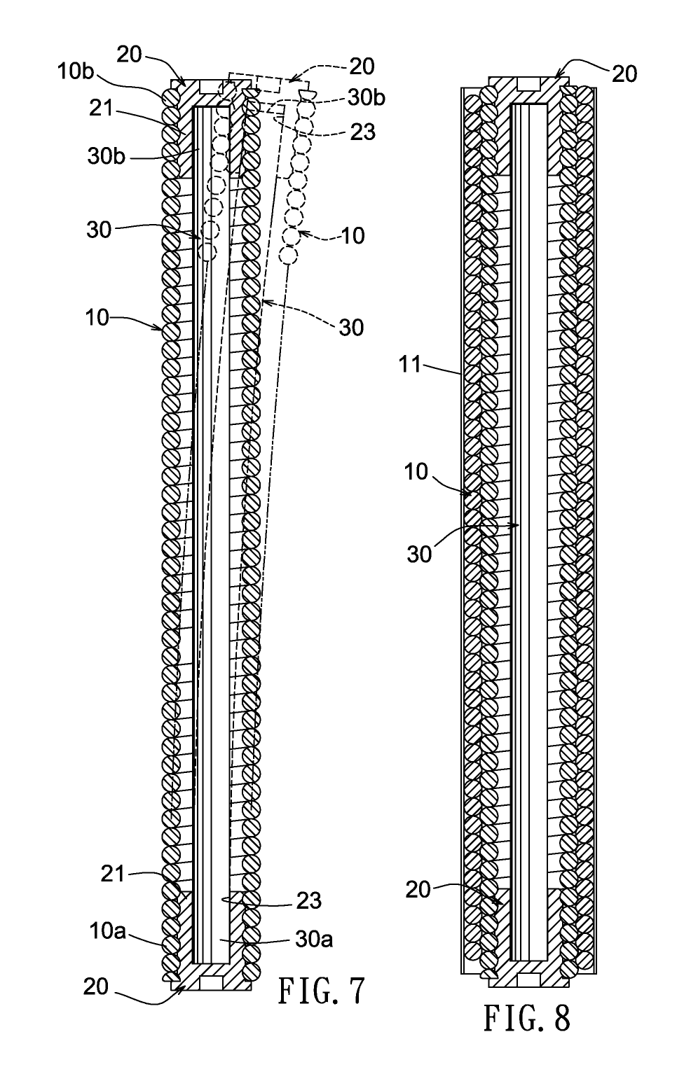

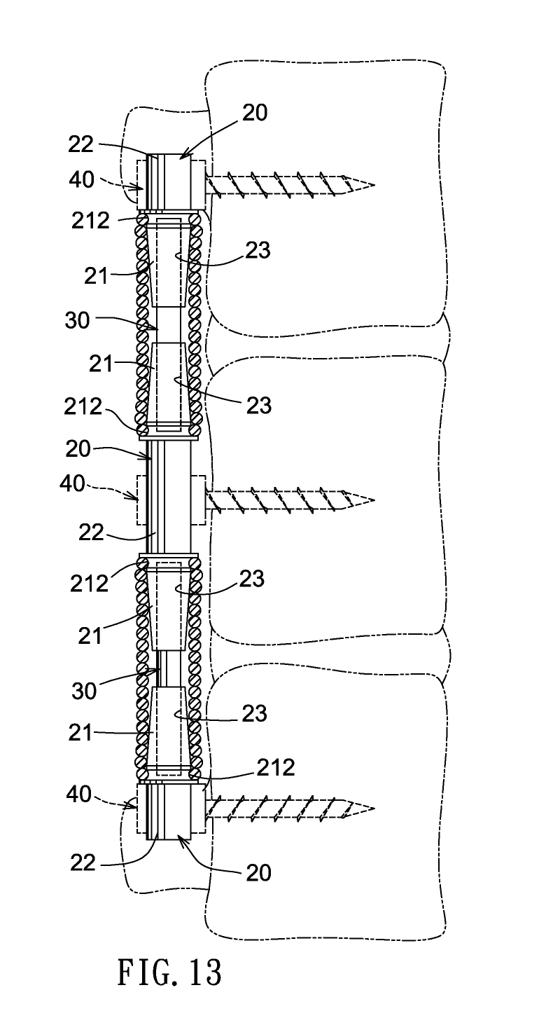

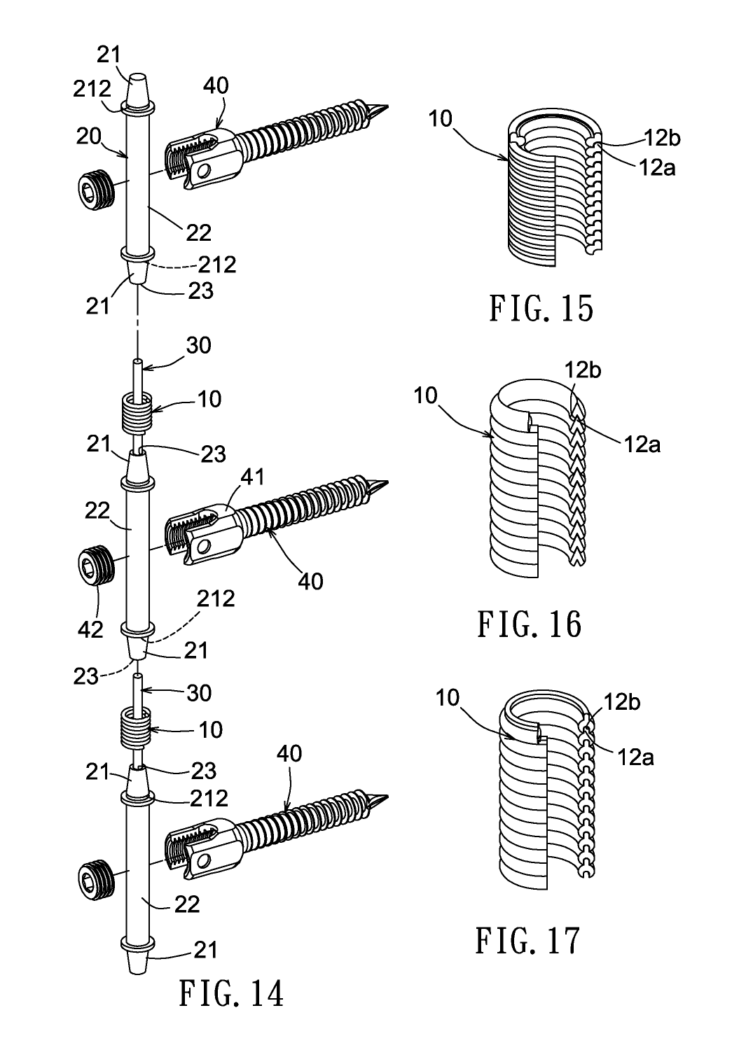

The coil spring member 10 of the spine stabilization device is made of a spiral spring wire having multiple continuous loops. The coil spring member 10 has the form of an elongated bar. Each connection member 20 has a connection section 21. The connection sections 21 are securely connected with two ends 10a, 10b of the coil spring member 10. The opposite end faces of the two connection members 20 are respectively formed with two axially extending sockets 23. The elastic bar 30 axially extends through the coil spring member 10 with two ends 30a, 30b of the elastic bar 30 respectively extending into the sockets 23 of the connection members 20. Even if the coil spring member 10 is axially tensioned to a maximum allowable length or bent to a maximum curvature as shown by the phantom lines of FIG. 7, the two ends 30a, 30b of the elastic bar 30 are still positioned in the sockets 23 of the connection members 20. As aforesaid, the coil spring member 10 is made of a spiral spring wire. Therefore, two ends of the coil spring member 10 are naturally formed with threads, which can be directly screwed on the corresponding spiral grooves 221 of the connection members 20. Alternatively, as shown in FIGS. 13 and 14, the threads can be elastically engaged with the annular grooves 212 of the connection members 20. According to the above arrangement, after the coil spring member 10 is formed, the coil spring member 10 can be securely connected with the connection members 20 without being further mechanically processed. This facilitates the manufacturing process to lower the manufacturing cost.

Moreover, in the anti-displacement coil spring-type spine stabilization device of the present invention, it is impossible to pull and multistage deflect the elastic bar 30 axially extending through the coil spring member 10. Therefore, the coil spring member 10 is prevented from being greatly displaced under radial action force. Also, the spiral loops of the coil spring member 10 are in tight contact with each other so that the coil spring member 10 cannot be compressed. In this case, the coil spring member 10 is able to resist against the axial compression force applied by the spine. Accordingly, the lesion vertebras can be effectively supported and released.

In application of the anti-displacement coil spring-type spine stabilization device of the present invention, the coil spring member 10 and the elastic bar 30 can be cut to a necessary length according to the requirement of use. After cut, the coil spring member 10 and the elastic bar 30 are installed and bridged over multiple vertebras 51 to protect the injured vertebras and intervertebral discs 52 of the spine 50. Accordingly, the present invention is flexibly adjustable in accordance with different spine structures of different patients to meet the requirements of the patients.

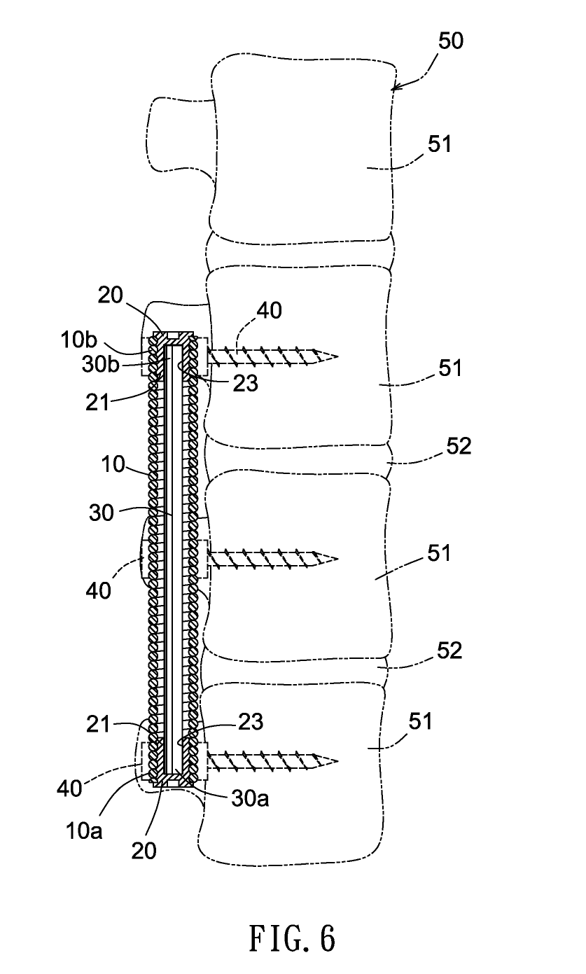

Referring to FIG. 6, in application of the anti-displacement coil spring-type spine stabilization device of the present invention, the pedicle screw 40 can be directly locked on any section of the coil spring member 10 without limitation. The pedicle screw 40 has a base seat 41 and a screw shank 411 protruding from a bottom of the base seat 41 for screwing into the spine 50. The base seat 41 is formed with a chuck 412. At least one rib 413 is formed on the chuck 412 for engaging between two adjacent loops of the coil spring member 10. A plug 42 is locked in the chuck 412 to tightly hold the coil spring member 10 between the chuck 412 and the plug 42. The pedicle screw 40 is not included in the scope of the present invention. Any other conventional pedicle screw is also applicable to the present invention without limitation.

In the anti-displacement coil spring-type spine stabilization device of the present invention, the pitch between the adjacent loops of the coil spring member 10 is equal, unequal, or nearly zero. That is, when the spine 50 bears an axial action force, the compressible distance is near zero. In this case, the lesion vertebras can be effectively supported and released. Moreover, the injured vertebras 51 or intervertebral discs 52 of the spine 50 are protected from being reinjured due to over-compression.

In the anti-displacement coil spring-type spine stabilization device of the present invention, the spring wire forming the coil spring member 10 can have a circular cross section, a rectangular cross section or a cross section of any other shape. Alternatively, as shown in FIGS. 15, 16 and 17, the opposite sides of the adjacent loops can be formed with raised section 12a and recessed section 12b engageable with each other. Accordingly, the adjacent loops are stacked with the raised sections 12a and the recessed sections 12b engaged or mated with each other without possibility of radial relative displacement. The raised section 12a and the recessed section 12b can be complementary rectangular raised section and recessed section, conic raised section and recessed section or any other raised section and recessed section with complementary shapes. The cross-sectional shape of the loops is not limited.

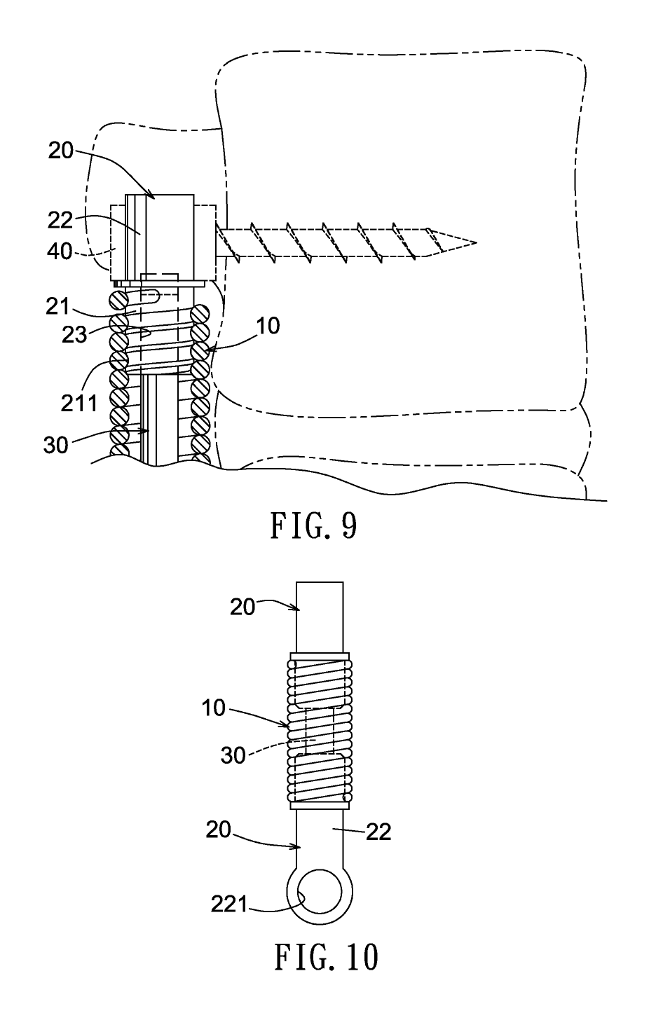

Referring to FIG. 9, in the anti-displacement coil spring-type spine stabilization device of the present invention, the connection member 20 connected with the end of the coil spring member 10 has a connection section 21. A rod body 22 axially extends from one end of the connection section 21. The rod body 22 is locked and connected with a pedicle screw 40. An end face of the connection section 21 is axially recessed to form at least one socket 23.

Referring to FIG. 10, the rod body 22 is formed with a shaft hole 221 for locking on a fixing shaft. Accordingly, the angle contained between the connection member 20 and the coil spring member 10 is adjustable in accordance with the requirement of installation of the anti-displacement coil spring-type spine stabilization device on the cervical vertebras and the occipital.

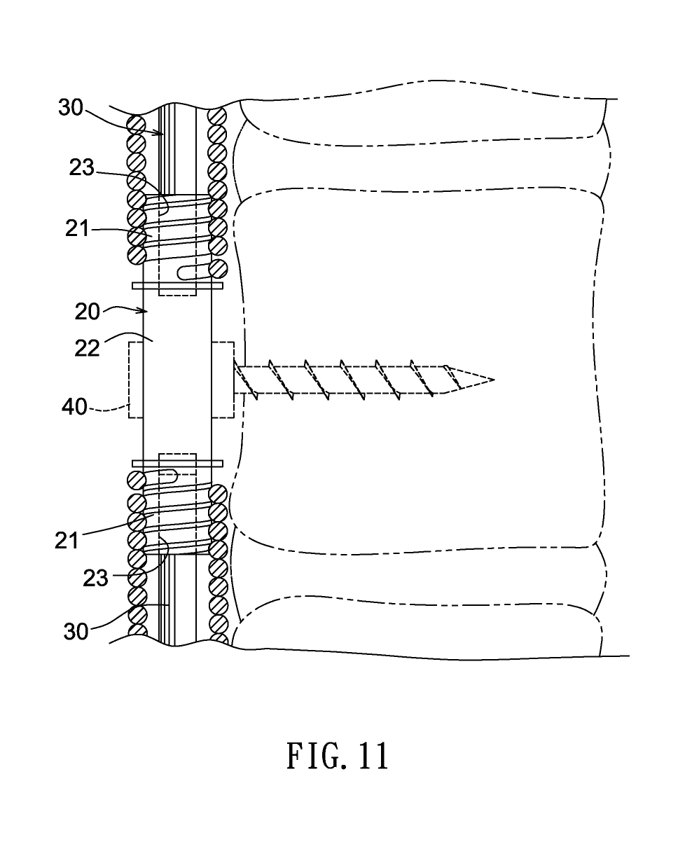

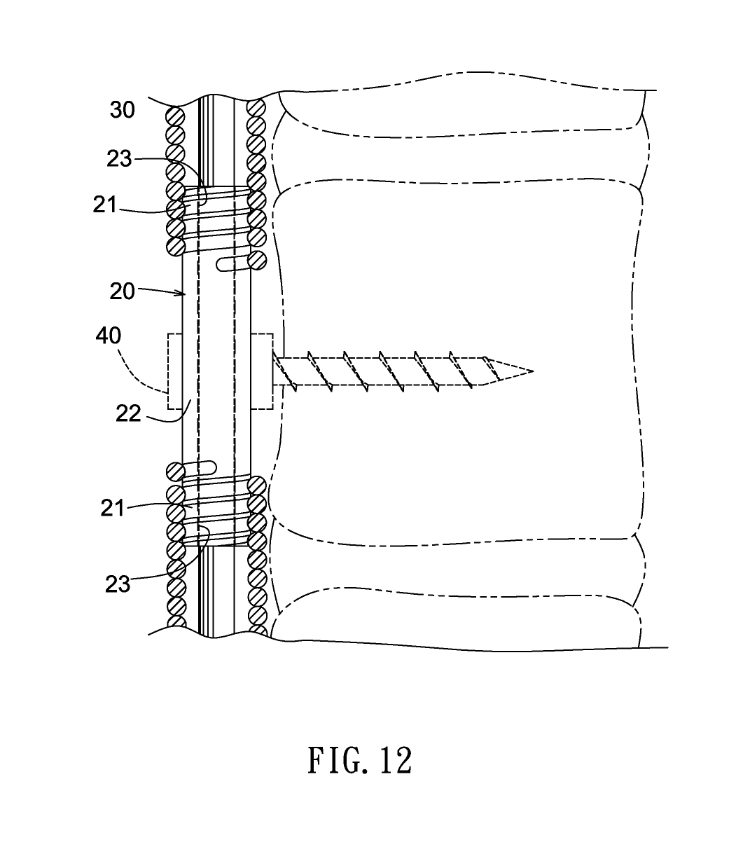

Referring to FIG. 11, in a modified embodiment, the connection member 20 has a rod body 22. A connection section 21 axially protrudes from each of two ends of the rod body 22 for connecting with the end of the coil spring member 10. The end face of the connection section 21 is axially recessed to form at least one socket 23. One end of the elastic bar 30 is fitted in the socket 23. In application of the present invention, the pedicle screw 40 is locked on the rod body 22 between the two connection sections 21 of the connection member 20. As shown in FIG. 12, the sockets 23 of the connection sections 21 at two ends of the connection members 20b can communicate with each other. In this case, the elastic bar 30 can pass through the connection members 20b and axially pass through multiple coil spring members 10.

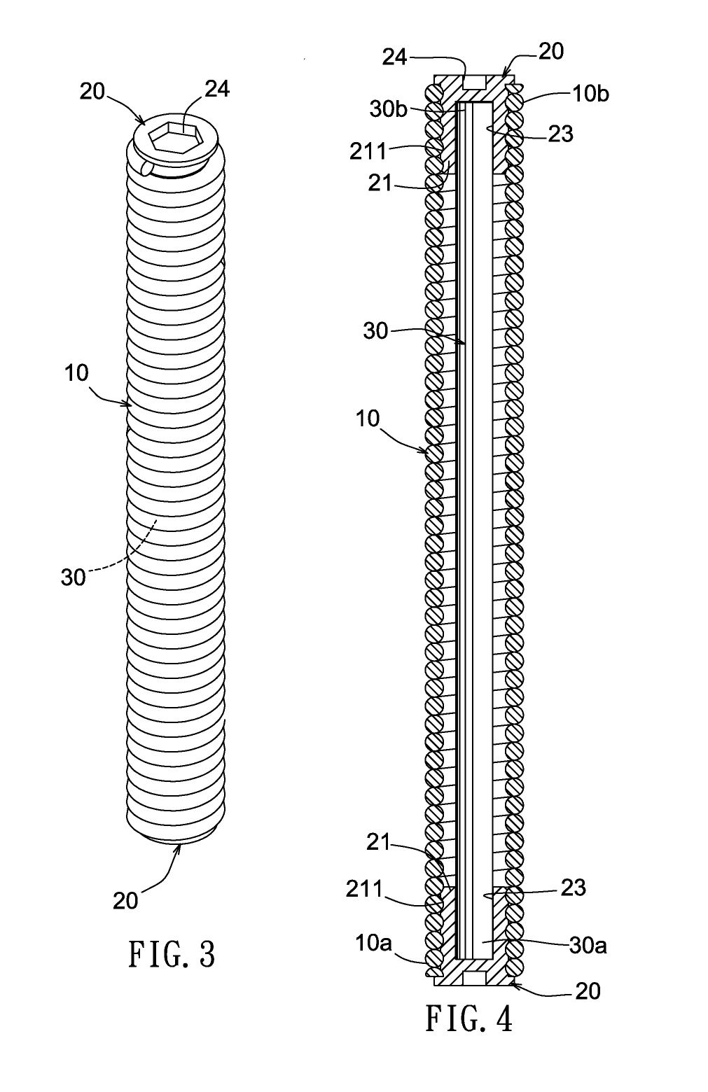

Referring to FIGS. 2, 9 and 12, the connection section 21 of the connection member 20 is formed with spiral groove 211 in adaptation to the spiral angle of the coil spring member 10. Accordingly, the connection member 20 can be snugly locked on the end of the coil spring member 10. As shown in FIGS. 2 and 3, the opposite ends of the connection sections 21 of the connection members 20 are formed with drive sections 24 for a wrench or a screwdriver to drive, for example, polygonal sockets, slots, cross recesses, polygonal protrusions or irregular protrusions. Accordingly, a wrench or a screwdriver can be used to drive the connection member 20 to make the connection section 21 tightly screwed in the end of the coil spring member 10. Alternatively, the opposite ends of the connection sections 21 of the connection members 20 can be formed with conic configuration.

Referring to FIGS. 13 and 14, the connection section 21 of the connection member 20 is formed with an annular groove 212. The loops of the coil spring member 10 are partially elastically latched in the annular groove 212 to securely connect the connection member 20 with the coil spring member 10.

The connection member 20 can be made of metal material, alloy material, plastic material or complex material. The material of the connection member 20 is not limited.

In the anti-displacement coil spring-type spine stabilization device of the present invention, the elastic bar 30 can be made of metal, carbon fiber, plastic or complex material such as PEEK. The material of the elastic bar 30 is not limited. The elastic bar 30 can be straight, curved or angled to meet the curve of human spine.

In the anti-displacement coil spring-type spine stabilization device of the present invention, the coil spring member 10 can be enclosed in a plastic or rubber sleeve 11 (as shown in FIG. 8) to prevent growing human muscle tissue from infiltrating into the coil spring member 10.

In the anti-displacement coil spring-type spine stabilization device of the present invention, the coil spring member 10 is, but not limited to, composed of multiple layers of continuous loops as shown in FIG. 8.

In the anti-displacement coil spring-type spine stabilization device of the present invention, two ends of the elastic bar 30 are movably fitted in the sockets 23 of the two connection members 20. Alternatively, one end of the elastic bar 30 is fixed in the socket 23 of one connection member 20, while the other end of the elastic bar 30 is movably fitted in the socket 23 of the other connection member 20.

In the anti-displacement coil spring-type spine stabilization device of the present invention, multiple elastic bars 30 can be fitted between two connection members 20. The number of the elastic bars 30 is not limited.

Referring to FIGS. 6 and 7, the coil spring member 10 is composed of multiple continuous loops and bendable and tensile. Also, the elastic bar 30 axially extending through the coil spring member 10 is elastically bendable along with the movement of the spine 50. Therefore, when the spine 50 of a patient equipped with the present invention moves, the coil spring member 10 of the present invention can swing or stretch along with the movement of the vertebras 51 of the spine 50 to simulate the movement of a normal spine tissue. This can relieve the patient from uncomfortableness.

As aforesaid, the coil spring member 10 of the present invention is composed of multiple continuous loops. Both the tensility and bendability of the coil spring member 10 are much better than that of a bar body cut with spiral grooves. With the anti-displacement coil spring-type spine stabilization device of the present invention, when a patient moves, the force will be transmitted to all the loops of the entire coil spring member 10 and the elastic bar 30. The coil spring member 10 will swing or stretch along with the vertebras 51 of the spine 50 to distribute the action force. Under such circumstance, the action force is prevented from concentrating on the vertebras that are not connected with the spine stabilization device. Accordingly, the healthy vertebras are protected from being injured due to over-forcing or over-use. Therefore, the injury caused by use of medical implements can be effectively lowered.

In the anti-displacement coil spring-type spine stabilization device of the present invention, the coil spring member 10 is formed of a spiral spring wire and the loops of the coil spring member 10 are in tight contact with each other. Therefore, it is nearly impossible to axially compress the coil spring member 10. In this case, the lesion vertebras can be effectively supported and released and thus the injured vertebras 51 or intervertebral discs 52 of the spine 50 are protected from being reinjured by axial compression force.

In the anti-displacement coil spring-type spine stabilization device of the present invention, the coil spring member 10 is formed of a spiral spring wire. After formed, the multiple loops of two ends of the coil spring member 10 will naturally define threaded holes or form outer threads, which can be snugly screwed on the corresponding spiral grooves 221 of the connection members 20. Alternatively, the loops can be directly elastically engaged with the annular grooves 212 of the connection members 20. According to the above arrangement, after the coil spring member 10 is formed, the coil spring member 10 can be securely connected with the connection members 20 without being further mechanically processed many times (such as cutting with the spiral groove, milling or tapping). This facilitates the manufacturing process to greatly lower the manufacturing cost of the present invention. Apparently, the present invention is advantageous over U.S. Publication No. US2007/0049937 A1 and U.S. Pat. No. 7,329,258.

The above embodiments are only used to illustrate the present invention, not intended to limit the scope thereof. Many modifications of the above embodiments can be made without departing from the spirit of the present invention.

What is claimed is:

1. An anti-displacement coil spring-type spine stabilization device, which is securely fixable on one side of a spine by multiple pedicle screws, the spine stabilization device comprising:
a coil spring member made of a spiral spring wire having multiple continuous loops, the coil spring member having the form of an elongated bar;
two connection members, each connection member having a connection section, the connection sections being formed with grooves for correspondingly latching with the loops of the coil spring member, whereby the connection sections are securely connected with two ends of the coil spring member, opposite end faces of the two connection members being respectively formed with two axially extending sockets; and
at least one elastic bar disposed in the coil spring member and axially extending through the coil spring member with two ends of the elastic bar respectively extending into the sockets of the connection members.

2. The anti-displacement coil spring-type spine stabilization device as claimed in claim 1, wherein the coil spring member is made of the spiral spring wire having multiple continuous loops in tight contact with each other, a pitch between each two adjacent loops of the coil spring member being nearly zero.

3. The anti-displacement coil spring-type spine stabilization device as claimed in claim 1, wherein opposite sides of cross sections of the adjacent loops of the spiral spring wire are formed with raised section and recessed section engageable with each other, whereby the adjacent loops of the coil spring member are stacked with the raised sections and the recessed sections engaged with each other.

4. The anti-displacement coil spring-type spine stabilization device as claimed in claim 1, wherein the connection member has a connection section, a rod body axially extending from one end of the connection section, the rod body being locked and connected with a pedicle screw.

5. The anti-displacement coil spring-type spine stabilization device as claimed in claim 4, wherein the rod body of the connection member is formed with a shaft hole for locking on a fixing shaft.

6. The anti-displacement coil spring-type spine stabilization device as claimed in claim 1, wherein the connection member has a rod body, a connection section axially protruding from each of two ends of the rod body for connecting with the end of the coil spring member, an end face of the connection section being axially recessed to form at least one socket.

7. The anti-displacement coil spring-type spine stabilization device as claimed in claim 6, wherein the sockets of the connection sections at two ends of the connection members communicate with each other.

8. The anti-displacement coil spring-type spine stabilization device as claimed in claim 1, wherein the groove formed on the connection section of the connection member is a spiral groove.

9. The anti-displacement coil spring-type spine stabilization device as claimed in claim 1, wherein the groove formed on the connection section of the connection member is an annular groove.

10. The anti-displacement coil spring-type spine stabilization device as claimed in claim 1, wherein the elastic bar is straight, curved or angled.

11. The anti-displacement coil spring-type spine stabilization device as claimed in claim 1, wherein the coil spring member is enclosed in a plastic or rubber sleeve.

12. The anti-displacement coil spring-type spine stabilization device as claimed in claim 1, wherein the coil spring member is composed of multiple layers of continuous loops.

13. The anti-displacement coil spring-type spine stabilization device as claimed in claim 1, wherein one end of the elastic bar is fixed in the socket of one of the connection members.

14. The anti-displacement coil spring-type spine stabilization device as claimed in claim 1, wherein multiple elastic bars are fitted between two connection members.

15. The anti-displacement coil spring-type spine stabilization device as claimed in claim 8, wherein opposite ends of the connection sections of the connection members are formed with drive sections for a wrench or a screwdriver to drive.
